(19) United States
(12) Patent Application Publication
(10) Pub. No.: US 2026/0335550 A1
(43) Pub. Date: Oct. 29, 2026

(54) ADD-ON DEVICE FOR AN INJECTION DEVICE

(71) Applicant: Sanofi, Paris (FR)

(72) Inventor: Michael Helmer, Frankfurt am Main (DE)

(21) Appl. No.: 19/107,397

(22) PCT Filed: Aug. 28, 2023
(86) PCT No.: PCT/EP2023/073460

§ 371 Date: Feb. 27, 2025

(30) Foreign Application Priority Data
Aug. 30, 2022 (EP) 22315201.8

(51) Int. Cl. A61M 5/20

(52) U.S. Cl. CPC A61M 5/20; A61M 2205/502; A61M 2205/60

(57) ABSTRACT

Disclosed is an add-on device for attaching to an injection device that includes a machine-readable identification. The add-on device includes a device body fastenable to a portion of the injection device, a reconfigurable and user-perceptible device identification, and an electronic module comprising a processor coupled to the device identification and operable to reconfigure the device identification in accordance with the machine-readable identification of the injection device.

### CROSS-REFERENCE TO RELATED APPLICATIONS

The present application is the national stage entry of International Patent Application No. PCT/EP2023/073460, filed on Aug. 28, 2023, and claims priority to European Application No. EP 22315201.8, filed on Aug. 30, 2022, the disclosures of which are incorporated herein by reference.

### TECHNICAL FIELD

The present disclosure relates to the field of add-on devices for injection devices, in particular, to add on devices attachable to a pen-type injector. In another aspect the disclosure relates to an injection system comprising an injection device and an add-on device. In further aspects the disclosure relates to a method of configuring an add-on device in the course of assembly to an injection device.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of equal or variable size. Moreover, a dose setting as well as a dose dispensing procedure must be easy to operate and has to be unambiguous.

A patient suffering from a particular disease may require a certain amount of a medicament to either be injected via a pen-type injection syringe.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule is always the same and does not change or cannot be changed over time.

Some injection devices are implemented as reusable injection devices offering a user to replace a medicament container, such as a cartridge. Other injection devices are implemented as a disposable injection device. With disposable injection devices it is intended to discard the entirety of the injection device when the content, i.e. the medicament, has been used up.

In order to control and to supervise administering of medication conducted by users or patients themselves it is beneficial to assist the user by making use of an external electronic device, such as a mobile electronic device, e.g. implemented as a smartphone, a tablet computer, or a smart watch. A software application provided on such external electronic devices may interact with the user and may provide instructions or recommendations to the user of how to correctly use the injection device.

In order to control and to supervise administering of medication conducted by users or patients themselves it is desirable to provide an automated detecting and logging of a repeated and regular use of the drug delivery device. A rather automated recording of doses injected by a user would offer a significant advantage over a manual dose logging in terms of security and convenience.

There exist numerous add-on devices configured for use with injection devices that offer an electronic detection and monitoring of repeated dose injection procedures.

Typically, such add-on devices or auxiliary devices can be detachably connected to an injection device. An add-on device typically comprises a detector or detector arrangement operable to detect a date and/or time when the user sets or injects a dose of the medicament. Some add-on devices also provide a quantitative measurement of a size of a dose currently set or dispensed. Some add-on devices are intended for use with a series of injection devices. This may particularly apply with disposable injection devices, that are intended to become discarded after use or after the medicament located therein has been used up.

Generally, there exists a variety of different injection devices, which may be equipped with different drugs or medicaments but are generally operable to be used with one and the same type of an add-on device. While it is common practice to characterize or to label injection devices and/or medicaments of different types and/or of different concentration by a clear and distinct label or identification the detachable connection with an add-on device may impose a disadvantage, in particular when a label or a characteristic portion of the injection device is covered by the add-on device when attached to the injection device.

Hence, when the injection device is equipped with an add-on device the label of the injection device being e.g. illustrative of the type of concentration of the medicament contained therein may be no longer visible or the visibility of such a label or identification may be compromised.

It is therefore desirable to set aside such disadvantages and to improve a clear and distinct labeling or characterizing of injection devices even when equipped with an add-on device.

### SUMMARY

In one aspect there is provided an add-on device for attaching to an injection device. The injection device comprises a machine-readable identification. The add-on device comprises a device body that is fastenable to a portion of the injection device. The add-on device further comprises a reconfigurable and user-perceptible device identification. The add-on device further comprises an electronic module comprising a processor coupled to the device identification and operable to reconfigure the device identification in accordance to the machine-readable identification of the injection device.

The add-on device is particularly adapted to mimic or to adapt to a differentiation or characterization of the injection device, which characterization is defined or reflected by the machine-readable identification thereof. The machine-readable identification of the injection device is typically implemented to distinguish the injection device and/or the medicament contained inside the injection device or dispensable by way of the injection device from another injection device and/or from another medicament. Hence, there may be provided multiple injection devices, each of which comprising the same or similar shape, purpose and/or way of operating, wherein each injection device is equipped with a different medicament, e.g. with medicaments of different concentrations and/or with different pharmaceutically active compounds.

The machine-readable identification is indicative of such device characteristics and/or medicament characteristics. With some examples a first injection device is equipped with a device portion of a first color. A second injection device may be provided with a comparable device portion but in a second color that distinguishes from the first color. Accordingly, the first injection device may be provided with a first machine-readable identification being indicative of the first color. The second injection device may be provided with a second machine-readable identification being indicative of the second color.

The reconfigurable and user-perceptible device identification is reconfigurable particularly before or during attaching the add-on device to the injection device. The user-perceptible device identification can be configured, reconfigured or deployed at least once in order to adapt the user perceptible device identification to the machine-readable identification of the injection device. This way, the user-perceptible device identification of the add-on device can be adapted to the machine-readable identification of the injection device to which the add-on device is or is to be attached to. This is of particular use when the add-on device, while attached to the injection device, covers a portion of the injection device that is typically characteristic for the injection device and/or for the medicament located therein.

A portion of the injection device covered by the add-on device and/or being provided with a user perceptible identification of the injection device can be effectively transferred to the add-on device so that after an attachment of the add-on device to the injection device a respective user perceptible device identification, e.g. being indicative of a characteristic of the injection device and/or of a property of the medicament located therein, is and remains perceptible to the user of the injection device equipped with the add-on device.

According to a further example the add-on device comprises at least one of a reader and a transceiver coupled to the processor and operable to read or to obtain the machine-readable identification of the injection device. The transceiver may be operable to read or to obtain the machine-readable identification directly when or before the add-on device is attached to the injection device. The reader may be implemented as an optical reader. The machine-readable identification may be physically provided on a portion of the housing of the injection device. The machine-readable identification may comprise a machine-readable code, such as an optical code, e.g. a barcode or a data matrix code. Here, the reader of the add-on device may comprise an imaging system, such as a camera operable to read e.g. the optical code of the machine-readable identification.

With further examples the machine-readable identification may comprise a wireless communication device, such as a wireless communication chip. The machine-readable identification may comprise at least one of an active or a passive RFID or NFC chip or tag operable to communicate with the transceiver of the add-on device.

This way, the electronic module may be configured to automatically set up or build up a communication link between the transceiver and the machine-readable identification as provided on or in the injection device thereby identifying the injection device and/or its characteristics. According to the machine-readable identification read or obtained from the injection device and transmitted by the transceiver of the add-on device to the processor of the electronic module of the add-on device the processor may appropriately process the obtained information and may be further operable to configure or to reconfigure the device identification.

The device identification is operable to generate a user perceivable identifier being indicative of the machine-readable identification of the injection device. With further examples the reader or transceiver of the add-on device is operable to indirectly obtain the machine-readable identification of the injection device. Here, the reader or transceiver of the add-on device may be operable to communicate with an external electronic device, such as a portable electronic device. The external electronic device may be operable to read or to obtain the machine-readable identification of the injection device and may be further operable to forward or to exchange this information to or with the add-on device. Hence, the add-on device may obtain the machine-readable identification of the injection device indirectly, via a communication link, e.g. a wireless communication link with an external electronic device, such as a smart phone or smart watch.

According to a further example at least one of the reader and the transceiver comprises a wireless near field transceiver operable to wirelessly communicate with the machine-readable identification of the injection device in order to read or to obtain information or data provided by or in the machine-readable identification of the injection device. Here, the machine-readable identification may be implemented as a near field communication chip, e.g. as a NFC chip or NFC tag. The transceiver or reader of the add-on device may be implemented accordingly. It may be implemented as a NFC reader operable to communicate with the NFC chip or NFC tag of the injection device providing the machine-readable identification.

A suitable pairing between the machine-readable identification of the injection device and the reader or transceiver of the add-on device may be automatically triggered as soon as the wireless near field transceiver of the add-on device gets in a predefined short range distance to the machine-readable identification of the injection device. With some examples the add-on device is attachable to a proximal end of the injection device, e.g. to a dial extension and/or to at least one of a dose dial and a trigger. Here, the machine-readable identification, in particular a short range identification tag of the injection device may be provided in or on the proximal end of the injection device. It may be provided on or in a proximal end face of a trigger or trigger button of the injection device.

With some examples the add-on device comprises a receptacle to receive a portion, e.g. the proximal end of the injection device. This way the machine-readable identification may be located inside the receptacle of the add-on device when the add-on device is suitably attached to the proximal end of the injection device.

By way of providing a wireless near field transceiver to the add-on device and by having a corresponding short range machine-readable identification on or in the injection device there can be provided a rather failure safe mutual coupling between the injection device and the add-on device only and exclusively when the add-on device is within a predefined spatial range to the injection device.

With some examples, and with the short range transceiver of the add-on device establishing or set up of a communication link between the machine-readable identification and the wireless near field transceiver of the add-on device may require a distance less than 5 cm, less than 2 cm, less than 1 cm or even less than 0.5 cm. In this way it can be provided that a reading of the machine-readable identification of the injection device is only possible when the add-on device with its device body is suitably fastened to a dedicated portion of the injection device. It is only upon a correct mounting of the add-on device to the injection device that the machine-readable identification can be suitably extracted and processed by the electronic module of the add-on device for configuring or reconfiguring the user perceptible device identification of the add-on device.

According to a further example the at least one of the reader and the transceiver comprises a wireless local range transceiver. The wireless local range transceiver is operable to wirelessly communicate with an external electronic device to receive the machine-readable identification of the injection device from or via an external electronic device. Here, the external electronic device, e.g. implemented as a smart phone, may be operable to read the machine-readable identification of the injection device and to forward the respective identification, hence the respective information content to the add-on device via the local range transceiver. The local range transceiver may use a communication protocol that differs from the communication protocol of the near field transceiver. Typically, the wireless local range transceiver comprises a transmission range that is larger compared to the transmission range of the wireless near field transceiver. Typically, the wireless local range transceiver can be implemented as a Bluetooth transceiver, as a Bluetooth low energy transceiver (BLE) or as a Wi-Fi transceiver.

This way the wireless local range transceiver may be used or may be usable to indirectly read out the machine-readable identification of the injection device, e.g. via a communication link with an external electronic device. The wireless local range transceiver of the add-on device may be further operable to communicate with the external electronic device, e.g. to synchronize or to exchange data between the add-on device and the external electronic device. It may be particularly configured to transmit data measured by the add-on device to the external electronic device for further processing.

The add-on device may also comprise a sensor or sensor assembly operable to detect or gather data being indicative of an operation of the injection device. The sensor may be operable to track or to measure a movement or configuration of a characteristic component of the injection device during its use. Data, such as injection data being indicative of an operation of the injection device and gathered or collected by the sensor may be transmitted to the external electronic device via the transceiver. The transceiver may be implemented as a Bluetooth transceiver or as a BLE transceiver.

The electronic module and hence the add-on device comprises a memory configured to store a plurality of measurement results of the sensor. The electronic module and hence the add-on device further comprises a clock configured to provide each of a plurality of measurement data of the sensor with a time index indicating the point of time and/or date of the detection of the respective operation of the injection device 1.

With some examples, the sensor further comprises an acceleration sensor configured to detect an injection operation of the injection device 1 and/or to detect or to classify gestures of a user when using the sensor and/or the injection device 1. For example, and when the injection device is implemented as an autoinjector, the acceleration sensor may detect an acceleration of a needle of the autoinjector upon executing a dispensing or injection procedure.

The sensor may further comprise a position sensor, which may be operable to detect the position or orientation of a dedicated component of the drive mechanism of the injection device. The position sensor may be operable to detect e.g. the position of a last dose nut, of a piston rod or the like component of the drive mechanism being indicative of the amount of medicament located inside the medicament container.

In addition and with further examples the electronic module and/or the sensor may comprise a microphone, by way of which characteristic click noises of the injection device can be detected and processed, thereby allowing to derive or to measure a size of a dose currently set or injected by the injection device.

Moreover, the external electronic device may wirelessly couple to the add-on device in order to configure the add-on device and/or in order to provide an extended operability or functionality of the add-on device to a user. There may be further provided a dialogue function between the add-on device and the external electronic device, e.g. via the wireless local range transceiver. By way of the dialogue function a user may configure or reconfigure any setting or configurations of the add-on device via the external electronic device.

With some examples the add-on device comprises both, a wireless near field transceiver and a wireless local range transceiver. Here, the add-on device may be operable to obtain and/or read the machine-readable identification of the injection device indirectly, e.g. via or by way of the wireless near field transceiver. By way of the wireless local range transceiver the add-on device may communicate with the external electronic device, such as a portable and/or mobile electronic device. Here, it is the electronic device that is operable to read the machine-readable identification of the injection device. The respective information is then transmittable to the add-on device, e.g. via the communication link between the add-on device and the external electronic device.

In this way, the add-on device is operable to autonomously read or to obtain the machine-readable identification from the injection device. Furthermore, the add-on device is operable to communicate with an external electronic device, even if the external electronic device is only in indirect vicinity to the injection device or add-on device. Typically, the local range transceiver comprises a transmission range in the region of a few meters.

According to a further example the device identification in a first configuration represents a first identifier being indicative of a first machine-readable identification. The device identification in a second configuration represents a second identifier being indicative of a second machine-readable identification. The first identifier and the second identifier perceptibly distinguish from each other. In this way and when the add-on device is fastened or attached to a first injection device equipped with a first machine-readable identification the device identification of the add-on device provides or represents the first identifier. Detaching of the add-on device and re-attaching the add-on device to another injection device, e.g. to a second injection device equipped with the second machine-readable identification that distinguishes from the first machine-readable identification provides a rather automated reconfiguration of the user perceptible device identification of the add-on device. Then and in the course of attaching the add-on device to the second injection device the device identification of the add-on device is operated or operable to generate and/or to provide the second identifier, which perceptibly distinguishes from the first identifier.

In this way the add-on device with its reconfigurable and user-perceptible device identification can be individually and suitably adapted to the injection device to which the add-on device is momentarily attached to.

According to a further example the device identification comprises an electronic signal generator controlled or operable by the processor. The electronic signal generator is operable to generate an electronic user perceivable signal. The electronic signal generator is particularly operable by the processor in accordance to the machine-readable identification as obtained or read by the reader or transceiver of the add-on device and as provided to the processor via the reader or transceiver of the add-on device.

According to a further example the electronic signal generator is operable to generate at least one of a visual, an acoustic and a haptic device identification and/or hence a respective identifier of visual, acoustic and/or haptic type.

With some examples, wherein the electronic signal generator is operable to generate at least a visual device identification or a respective identifier, various device identifications or various identifiers may distinguish by at least one of a color, a brightness and/or a temporal variation, e.g. in form of a blinking or flashing light.

When the electronic signal generator is implemented to generate an acoustic signal, the signals may distinguish by at least one a frequency, a volume and a temporal variation. When implemented as a haptic device identification or as a haptic identifier the electronic signal generator may be typically implemented as a kind of a vibration generator, e.g. operable to generate vibration signal that distinguish in strength, vibration frequency and/or by their temporal variation.

Any user perceptible device identification or a respective identifier that can be generated by the electronic signal generator may be characteristic for a particular machine-readable identification of the add-on device. For instance, and for different types of medicaments there may be provided different types of machine-readable identifications on or in the injection device. For each one of these different medicaments there may be provided a unique device identification and/or a respective identifier that distinguish with regard to their color. With some examples the electronic signal generator comprises at least a first and a second light source, wherein the first and the second light sources are operable to emit light of different colors and/or frequency.

According to a further example the electronic signal generator comprises at least two light sources of different color to generate device identifications or generated device identifications that distinguish with regards to their color.

The electronic signal generator may further comprise a kind of a light guide or an illumination surface optically coupled to the first and second light sources. Now and for providing different user-perceptible device identifications and/or in order to provide different identifiers with the signal generator there may be activated only one of the first and the second light sources or the other one of the first or second light sources, thereby providing identifiers and/or user-perceptible device identifications of different colors. By activating both light sources there may be provided a mixed color and hence a third color, representing a third identifier that distinguishes from the colors of the first light source and the second light source.

By increasing the number of light sources there can be provided a large variety of differently color encoded user perceptible device identifications or respective identifiers.

In the same way there can be provided respective acoustic signals, such as characteristic sounds or jingles that are associated to the characteristics of different injection devices and/or different medicaments to be injected with such devices. In the same way there can be generated characteristic haptic signals by way of a vibration generator. With acoustic and haptic device identifications a characteristic encoding of the add-on device can be provided, e.g. by generating characteristic sounds featuring respective acoustic or haptic signals of varying duration and/or magnitude.

With some examples the electronic signal generator may activate anonymously, e.g. when the add-on device is transferred from a sleep mode into an activated mode. This way and every time a user intends to use the injection device equipped with the add-on device the respective user-perceptible device identification will be generated or refreshed and provided to the user.

With some examples and when generation of the identifier or of the user perceptible device identification consumes electric energy the electronic signal generator may automatically switch off or may be transferred into a sleep mode, e.g. after lapse of a predefined time interval the add-on device has been used the last time.

According to a further example the add-on device comprises an electronic display coupled to the processor and operable to illustrate the device identification. Here, the electronic display may comprise a display section that can be reconfigured in order to provide or to act as a user-perceivable device identification. Here, the user-perceptible device identification may be provided by the electronic display of the device. Here, a background color of the electronic display may be representative of the machine-readable identification of the injection device.

With other examples a particular or characteristic display section may represent the user-perceptible device identification. For instance, and when the user perceptible device identification is provided by a characteristic color, the information as usually provided on or by the electronic display may be reproduced in this particular color-code or an information content as provided on the electronic display may be framed by the characteristic color-code of the injection device or medicament as encoded by the machine-readable identification thereof.

With further examples it is conceivable that the electronic display comprises an electrophoretic display exhibiting a particularly low electric power consumption, in particular when the add-on device is not in use. Such an electronic display may persistently or durably represent or provide the user-perceptible device identification, e.g. in form of a specific color and/or in form of a characteristic pattern or illustration on the electronic display.

According to a further example the device identification is persistently and/or durably perceivable from outside the add-on device. The device identification and/or the respective identifier may remain persistently and durably on the add-on device until it is reconfigured by the processor of the electronic module, e.g. in the course of detaching and re-attaching the add-on device to another injection device.

With some examples the electronic module, in particular its processor may be configured to configure or to reconfigure the device identification when detecting a machine-readable identification of the injection device, e.g. in the course of assembling or attaching the add-on device to the injection device.

With a further example the electronic module and/or the processor may be operable to deactivate the device identification when contact to the machine-readable identification of the injection device is lost, e.g. in the course of detaching the add-on device from the injection device. Hence, in an initial configuration the user perceptible device identification may be deactivated or may represent a kind of a neutral identification being void of an identifier that would be mapped to a particular injection device. It may be then only and exclusively upon establishing a connection or an attachment of the add-on device to the injection device that the machine-readable identification of the injection device is read out and that the processor of the electronic module of the add-on device configures or reconfigures the user perceptible device identification in accordance to the data obtained from the machine-readable identification of the injection device.

According to a further example the device identification is arranged on or is integrated into the device body. With some examples the device identification may comprise a window located in a sidewall of the device body. The window may cover an electronic signal generator operable to generate at least a visual device identification and/or a visual identifier. The user perceptible device identification may comprise a kind of a light guide optically coupled with the light source or with numerous light sources of the electronic signal generator.

In use the add-on device and in particular the device identification arranged on or integrated into the device body may illuminate in a characteristic color being indicative of a particular injection device or medicament.

Integration of the device identification into the device body may provide a rather robust and compact design of the add-on device. Providing or integrating the device identification in or on the device body is also rather intuitive for the user to recognize a characteristic device identification or identifier.

According to a further example the add-on device comprises a movable part operably coupleable with a trigger of the injection device and movable relative to the device body. Here, the device identification is arranged on or is integrated into the movable part. With some examples the movable part of the add-on device may protrude from a proximal end of the device body. The movable part may be implemented as a kind of an auxiliary injection button or trigger button, which is to be depressed and to be moved in distal direction relative to the device body in order to initiate or to start a dispensing action of the injection device when the add-on device is suitably attached to the injection device.

When the movable part is operably coupled with the trigger of the injection device a dispensing force applied to the movable part relative to the device body may initiate a respective movement of the trigger relative to a housing of the injection device thereby initiating a dispensing operation of the injection device. By implementing the user-perceptible device identification in or on the movable part there can be provided a rather well-defined and easily perceptible indication to the user being indicative of the particular type of the injection device to which the present add-on device is attached to.

With some examples the movable part comprises a window, a transparent portion or a partially transparent, hence a semitransparent portion, through which perceivable signals of the electronic signal generator are perceptible, e.g. visible. With further examples the entire movable part may be made of a transparent or semitransparent material, e.g. a semitransparent plastic material. In combination with an electronic signal generator comprising at least one or several light sources the movable part may comprise or constitute a signaling device configured to visually indicate the machine-readable identification of the injection device.

With some examples and in a first configuration of the device identification the movable part of the add-on device may be illuminated in a first color, e.g. in red. With another configuration of the user-perceptible device identification the movable part may be illuminated in a different color, e.g. in green.

With further examples the electronic signal generator may be operable to generate a visible signal defined by a sequence of light pulses or illumination pulses. The electronic signal generator, in particular its light source(s) may be operable to generate an encoded or a characteristic sequence of light pulses or illuminating pulses that may be visible at the proximal end of the add-on device, e.g. at or in the movable part.

By implementing the movable part as a transparent or semitransparent component of the add-on device the user-perceptible device identification can be rather elegantly integrated into the add-on device. An existing component of the add-on device, originally intended for operating the add-on device can be thus provided with a second function, namely with a signaling or identification function. Moreover, the add-on device provided with a movable part implemented as a signaling device provides a rather attractive design and improves an intuitive handling of the add-on device, especially when coupled with the injection device. This way, user attractivity and user acceptance of the add-on device can be further enhanced.

In a further aspect the present disclosure relates to an injection system. The injection system comprising an injection device for setting and injecting a dose of a medicament. The injection device comprises a machine-readable identification. The machine-readable identification is indicative of one of a type of the injection device and the type of the medicament. The injection system further comprises an add-on device as described above detachably attachable to the injection device. The machine-readable identification may be provided on or in a housing of the injection device. The machine-readable identification may comprise one of an optical or visible identification and an electronically readable identification. An electronically readable identification may comprise a communication chip, such as a RFID or NFC tag. The machine-readable identification may be implemented as a passive or as an active electronic component, being void of a power source or being equipped with an own power source, respectively.

When implemented as a passive electronic component the machine-readable identification is operable to withdraw or to obtain electrical energy from an interrogating RF field, e.g. provided by a reader or transceiver of the add-on device. The electronic machine-readable identification may be operable to communicate with a reader or transceiver of the add-on device in order to transfer an identification information of the injection device to the add-on device.

With some examples the injection device comprises a primary medicament container, e.g. in form of a syringe or in form of a cartridge. The syringe or cartridge typically comprises a tubular-shaped barrel sealed in a proximal direction by a movable stopper. The movable stopper is to be displaced or urged towards a distal direction, e.g. by way of a piston rod of a drive mechanism of the injection device. This way a dose of the medicament or multiple doses of the medicament can be dispensed through a distal outlet of the primary medicament container.

With some examples the machine-readable identification is provided on or in the primary medicament container. It may be provided in or on a label attached to an outside surface of the barrel of the cartridge or syringe.

With further examples the machine-readable identification may be provided on or in the stopper of the medicament container. With further examples the machine-readable identification is provided on an outside surface of a housing of the injection device. This way, the machine-readable identification may comprise a visible code, such as a barcode or as a matrix code. Here, the machine-readable identification may be identified through or via an imaging system, e.g. of an external electronic device. The external electronic device, e.g. implemented as a smartphone, as a smart watch or a tablet computer may be further operable to pair with the add-on device. This way, a configuration or reconfiguration of the add-on device may be conducted on the basis of the machine-readable identification, which is gathered or obtained by the external electronic device and which is transmitted to the add-on device via a separate communication link between the add-on device and the external electronic device.

With a further example the injection device comprises a dose dial and a trigger, e.g. provided at a proximal longitudinal end of the injection device. The injection device may be implemented as a pen-type injector. The dose dial and/or the trigger may be provided at a proximal longitudinal end of the pen-type injector. Here, the machine-readable identification may be provided in or on the dose dial and/or the trigger. The add-on device may be configured for releasable or detachable fastening to the proximal end of the injection device.

The add-on device, in particular its device body may comprise a receptacle to fit onto the dose dial and/or trigger. By implementing or arranging the machine-readable identification in or on the trigger or dose dial a geometric distance between the machine-readable identification and a reader or transceiver of the add-on device for readout of the machine-readable identification can be reduced to a minimum thus allowing to implement the transceiver or reader of the add-on device as a wireless near field transceiver operable to read a respective near field communication tag of the machine-readable identification of the device. This way, readout of the machine-readable identifier can only take place when the add-on device is correctly mounted to the proximal end of the injection device.

According to a further example the injection system comprises an external electronic device, e.g. implemented as a smartphone, a smart watch or as a tablet computer. The external electronic device is operable to read the machine-readable identification of the injection device. The external electronic device is further operable to transmit the identification of the injection device to the add-on device.

This way, the add-on device may be void of an own reader or transceiver operable to read the machine-readable identification of the injection device. Rather, it may be sufficient when the add-on device is equipped with only one transceiver, such as a wireless local range transceiver operable to wirelessly communicate with the external electronic device. Readout of the machine-readable identification of the injection device may be then provided and conducted through the external electronic device, which, when suitably paired with the add-on device is operable to provide the identifying information of the injection device to the add-on device, which then in turn may conduct a configuration or reconfiguration of the user perceptible device identification.

With this example the machine-readable identification of the injection device can be implemented in a variety of different ways. Here, the machine-readable identification may be implemented as a physical or optical code, which is readable or recognizable by an imaging system of the external electronic device.

With further examples the machine-readable identification comprises one of a mechanical code or mechanical encoding readable or detectable by the add-on device in the course of attaching the add-on device to the injection device.

With further examples the machine-readable identification comprises a magnetic code or magnetic encoding readable or detectable by the add-on device in the course of attaching the add-on device to the injection device.

With a further example the machine-readable identification comprises an electrical or electrostatic code our respective encoding readable or detectable by the add-on device in the course of attaching the add-on device to the injection device.

According to a further aspect the present disclosure relates to a method of configuring an add-on device. The method comprises the steps of providing an injection device, wherein the injection device comprises a machine-readable identification. The machine-readable identification comprises characteristic information of the injection device and/or of the medicament to be dispensed by the injection device. In a further step there is provided an add-on device as described above. The add-on device is configured to detachably fasten or to detachably connect to the injection device. Before, during or after attaching the add-on device to the injection device the machine-readable identification is read or gathered. In a further step the user perceptible device identification of the add-on device is configured or reconfigured in accordance to the machine-readable identification.

With some examples reading of the machine-readable identification is conducted by the add-on device itself. For this, the machine-readable identification may comprise a wireless short range communication tag, such as a NFC tag and the add-on device may comprise a corresponding wireless near field transceiver or near field reader operable to read-out information of the machine-readable identification.

With other examples the add-on device is operable to communicate with an external electronic device. Here, the add-on device may pair with the external electronic device and establish of a communication link with the external electronic device operable to transmit data between the add-on device and the external electronic device. With this example readout of the machine-readable identification may be conducted by the external electronic device. Information regarding the injection device and/or regarding the medicament as obtained by the readout of the machine-readable identification may be then transmitted from the external electronic device to the add-on device, which in turn may then reconfigure the user-perceptible device identification in accordance to the machine-readable identification.

Generally, the method of configuring the add-on device makes use of the add-on device as described above. Insofar, all features, effects and benefits described above in connection with the add-on device equally apply to the method of configuring the add-on device; and vice versa.

With a further aspect the present disclosure also relates to a computer program comprising computer readable instructions, which when executed by a processor of an add-on device cause the processor to reconfigure the user perceptible device identification of the add-on device in accordance to a machine-readable identification of injection device.

Typically, the computer program is executable by an add-on device as described above. It is further operable to conduct a method of configuring the add-on device as described above. Insofar, the computer program and its computer readable instructions are operable to cause a processor of the add-on device to conduct a method of configuring the add-on device as described above. The computer readable instructions may be executable by a processor of an add-on device as described above.

Additionally, or alternatively and when an external electronic device of an injection system as described above is configured to read-out the machine-readable identification of the injection device the computer program and its computer readable instructions may be at least in part executable by a processor of the external electronic device.

The computer program is to be executed by a processor of an add-on device as described above in order to execute a method of configuring the add-on device as described above. Insofar, all features, effects and benefits as described above in connection with the add-on device and/or described in connection with the method of configuring the add-on device as well as any effects, features and benefits as described above in connection with the injection system equally apply to the computer program; and vice versa.

The present disclosure further discloses and proposes a computer program including computer-executable instructions for performing the method according to the disclosed method/device/system in one or more of the examples enclosed herein when the program is executed on a processor, computer or computer network. Specifically, the computer program may be stored on a computer-readable data carrier. Thus, specifically, one, more than one or even all of the method steps as indicated above may be performed by using a computer or a computer network, typically by using a computer program.

The present disclosure further discloses and proposes a computer program product having program code means, in order to perform the method according to the disclosed method/system in one or more of the embodiments enclosed herein when the program is executed on a computer or computer network. Specifically, the program code means may be stored on a computer-readable data carrier.

Further, the present disclosure discloses and proposes a data carrier having a data structure stored thereon, which, after loading into a processor, computer or computer network, such as into a working memory or main memory of the processor, computer or computer network, may execute the method according to one or more of the examples disclosed herein.

The present disclosure further proposes and discloses a computer program product with program code means stored on a machine-readable carrier, in order to perform the method or parts thereof according to one or more of the examples disclosed herein, when the program is executed on a processor, computer or computer network. As used herein, a computer program product refers to the program as a tradable product. The product may generally exist in an arbitrary format, such as in a paper format, or on a computer-readable data carrier. Specifically, the computer program product may be distributed over a data network.

With another example, the present disclosure proposes and discloses a modulated data signal which contains instructions readable by a processor, a computer system or computer network, for performing the at least parts of a method according to one or more of the examples disclosed herein. Preferably, referring to the computer-implemented aspects of the disclosure, one or more of the method steps or even all of the method steps of the method according to one or more of the examples disclosed herein may be performed by using a processor, a computer or computer network. Thus, generally, any of the method steps including gathering, provision and/or manipulation of data may be performed by using a processor, a computer or computer network. Generally, these method steps may include any of the method steps, typically except for method steps requiring manual work, such as providing the samples and/or certain aspects of performing the actual measurements.

Specifically, the present disclosure further discloses a computer or computer network comprising at least one processor, wherein the processor is adapted to perform the method according to one of the examples described in this description, a computer loadable data structure that is adapted to perform the method according to one of the examples described in this description while the data structure is being executed on a processor, a computer, a computer program, wherein the computer program is adapted to perform the method according to one of the embodiments described in this description while the program is being executed on a computer.

Generally, the scope of the present disclosure is defined by the content of the claims. The disclosure is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20 °C), or refrigerated temperatures (e.g., from about −4 °C to about 4 °C). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom. Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### BRIEF DESCRIPTION OF THE DRAWINGS

In the following, numerous examples of a data logging device for monitoring use of an injection device as well as a respective injection device will be described in greater detail by making reference to the drawings, in which:

FIG. 1 schematically illustrates an example of an injection device,

FIG. 2 shows the process of assembling an add-on device to a proximal end of the injection device,

FIG. 3 shows three different configurations of an add-on device attached to the proximal end of the injection device,

FIG. 4 shows three further configurations of another example of an add-on device attached to the proximal end of an injection device,

FIG. 5 shows an example of the injection system,

FIG. 6 shows another example of an add-on device when attached to an injection device,

FIG. 7 shows three different examples of add-on devices, which are each illustrated in a first configuration and in a second configuration, respectively,

FIG. 8 shows a particular configuration of an external electronic device,

FIG. 9 shows a mutual pairing between the add-on device and the external electronic device,

FIG. 10 illustrates a readout of the machine-readable identification of the injection device in the course of assembling the add-on device to the injection device,

FIG. 11 shows a configuration of the external electronic device after a successful mutual pairing between the add-on device, the injection device and the external electronic device,

FIG. 12 is illustrative of an example of operation of the add-on device when attached to the injection device,

FIG. 13 shows a further example of operation of the add-on device when attached to the injection device,

FIG. 14 shows a block diagram of the add-on device, the injection device and the external electronic device and

FIG. 15 shows a flowchart of a method of configuring the add-on device.

### DETAILED DESCRIPTION

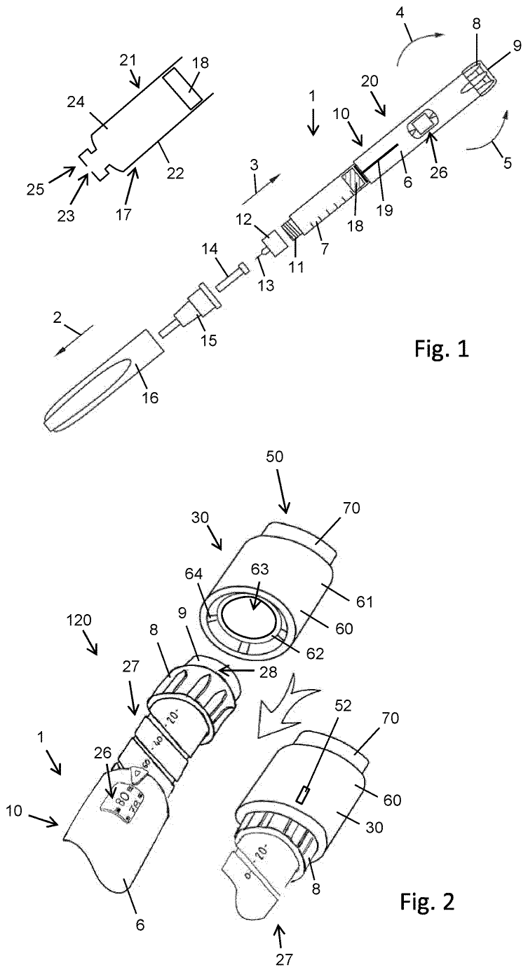

FIG. 1 shows an example of a drug delivery device 1, which is implemented as a handheld injection device. The injection device 1 may comprise or may be implemented as a pen-type injector. It may be implemented as a disposable injection device or as a reusable injection device. With some examples the injection device 1 is implemented as an autoinjector. The injection device 1 is of elongated shape. It may extend along a longitudinal direction. Towards a longitudinal distal direction 2 the drug delivery device 1 comprises a dispensing end for dispensing or injecting the medicament 24. Towards the proximal direction 3 the injection device 1 comprises at least one of a dose member 8 and a trigger 9, by way of which a dose of equal or individual or different size can be set and dispensed, respectively.

The injection device 1 comprises a housing 10. The housing 10 may comprise numerous housing components, such as a body 6 and a cartridge holder 7. The body 6 may be sized and configured to accommodate a drive mechanism 20. The cartridge holder 7 is sized and configured to accommodate a medicament container 21, e.g. implemented as a cartridge containing the liquid medicament 24. The medicament container 21 comprises a tubular-shaped barrel 22 sealed towards the distal end by a seal 23. The seal 23 may comprise a pierceable septum fixed to an outlet 25 of the medicament container 21. Towards a proximal end the interior of the barrel 22 is sealed by a piston 18 or stopper, which is slidably disposed inside the barrel 22.

By advancing the piston 18 towards the distal direction 2 a dose of the medicament 24 can be expelled from the medicament container 21. In use the medicament container 21 is arranged inside the cartridge holder 7. The drive mechanism 20 of the injection device 1 comprises a piston rod 19, which is displaceable in distal direction 2 for advancing the piston 18 towards the outlet 25 of the medicament container 21. Details of the drive mechanism are not further illustrated and described here. With some examples, the drive mechanism 20 may be implemented as an all-mechanical drive mechanism, where a user has to provide an entirety of a dispensing force required to move the piston rod 19 and hence the piston 18 in distal direction 2. With other examples, the drive mechanism comprises a mechanical energy storage configured to provide at least a portion of the dispensing force. Examples of drive mechanisms can be found e.g. in WO 2004/078241 A1, WO 2014/033197 A1 or WO 2014/033195 A1 the entirety of which are herein incorporated by reference.

With some examples, as e.g. described or shown in FIG. 2 the injection device 1 and hence the drive mechanism 20 may comprise a dial extension 27, which projects and moves in proximal direction 3 from a proximal end of the body 6 when or during setting a dose and which returns into its initial distal end position during a dose injection procedure. For this, a user may use a thumb 114 of his hand 110 to exert a distally directed pressure onto the trigger 9 thereby urging the dial extension 27 in distal direction 2 during a dose injection procedure.

For setting or dialing of a dose a user may twist or rotate the dose dial 8, e.g. in a dose incrementing direction 4, hence in a clockwise sense as seen from the proximal end. For correcting a dose previously set the user may also rotate the dose dial 8 in an opposite dose decrementing direction 5. The size of the dose is typically illustrated in a window 26 provided in or on the body 6 of the injection device 1. Prior to inject a dose of the medicament 24 the distal end of the cartridge holder 7 has to be connected with a needle assembly 12. For this, the distal end of the cartridge holder 7 comprises a connector 11, e.g. in form of a threaded interface to engage with a complementary shaped threaded counter interface of the needle assembly 12.

The needle assembly 12 is detachably or releasably fixable to the cartridge holder 7. It comprises a double-tipped injection needle 13. A proximal end of the injection needle (not shown) is configured to enter into a through opening at the distal end face of the connector 11 or cartridge holder 7 so as to pierce or to penetrate the seal 23 of the medicament container 21. The distal end of the injection needle 13 is typically covered by a detachable inner needle cap 14. The entirety of the needle assembly 12 may be covered by a detachable outer needle cap 15.

The cartridge holder 7 and hence a portion of the housing 10 is to be received in a protective cap 16, which is detachably connectable to the cartridge holder 7 or body 6.

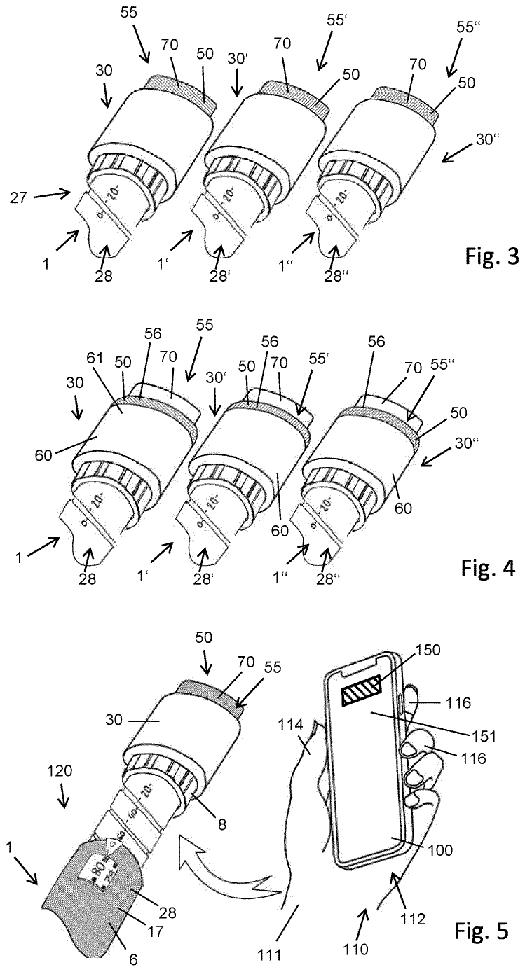

In FIGS. 2 and 3 there is shown an example of an add-on device 30 configured for fastening to the proximal end of the injection device 1. The add-on device 30 comprises a sensor 48 or sensor assembly, which is operable to detect, to recognize, to characterize and/or to measure an operation of the injection device 1.

The add-on devices 30 as illustrated in the various FIGS. 2-14 is detachably connectable to the dose dial 8. It comprises a device body 60 with a tubular-shaped sidewall 61. Towards the distal end the sidewall 61 confines a receptacle 63, which is sized to receive the dose dial 8 and the trigger 9 of the injection device 1. For this, the inside of the sidewall 60 may comprise one or numerous fastening ribs 64, which are configured to provide a slip free fastening of the add-on device 30 to the dose dial 8.

The device body 60 comprises the receptacle 63 at a distal end section of the add-on device 30. The receptacle 63 is open towards the distal direction 2. It is sized and configured to fit onto the proximal end of the injection device 1. An inside surface of the sidewall 61 confining the receptacle 63 comprises numerous fastening ribs 64, e.g. of an elastic or elastically deformable material, such as an elastomeric material.

The fastening ribs 64 provide a friction fit with the dose dial 8 when the device body 60 is assembled to the dose dial 8 of the injection device 1. The receptacle 63 may be confined in longitudinal direction by a flange portion 62 protruding radially inwardly from the tubular-shaped sidewall 61 of the receptacle 63. The flange portion 62 is located proximally offset from the insert opening of the receptacle 63. The flange portion 62 may be of annular shape and may be configured to axially abut on a stepped down section at the proximal end of the dose dial 8. Hence, the trigger 9, which comprises a reduced diameter compared to the dose dial 8 may protrude in proximal direction 3 through the flange portion 62 while the flange portion 62 may rest or abut against the proximal end face of the dose dial 8 when the add-on device 30 is suitably fastened to the dose dial 8.

The add-on device 30 further comprises a movable part 70 protruding in proximal direction from the device body 60. The movable part 70 comprises or forms an auxiliary trigger or trigger button, which is configured to mechanically engage with the trigger 9 of the injection device 1 when the add-on device 30 is correctly assembled to the injection device 1.

Typically, the add-on device 30, in particular, the device body 60 is frictionally engageable with the dose dial 8. In this way, a user may apply a dose setting torque onto the dose dial via the device body 60. Instead of rotating the dose dial 8 for setting of a dose the user may simply rotate the device body 60 relative to the body 6 of the injection device 1. This way, the dial extension 27 may become subject to a dose incrementing dialing or rotating motion. A respective size of a dose currently set will then be displayed in the window 26 of the body 6 of the injection device 1 as illustrated in FIG. 2.

For dispensing of a dose the user has to depress the movable part 70, which may then be subject to a distally directed motion relative to the device body 60. The movable part 70, which may be in direct or indirect mechanical engagement with the trigger 9, may then apply a respective dispensing force onto the trigger 9 thereby initiating a dose dispensing action of the injection device 1.

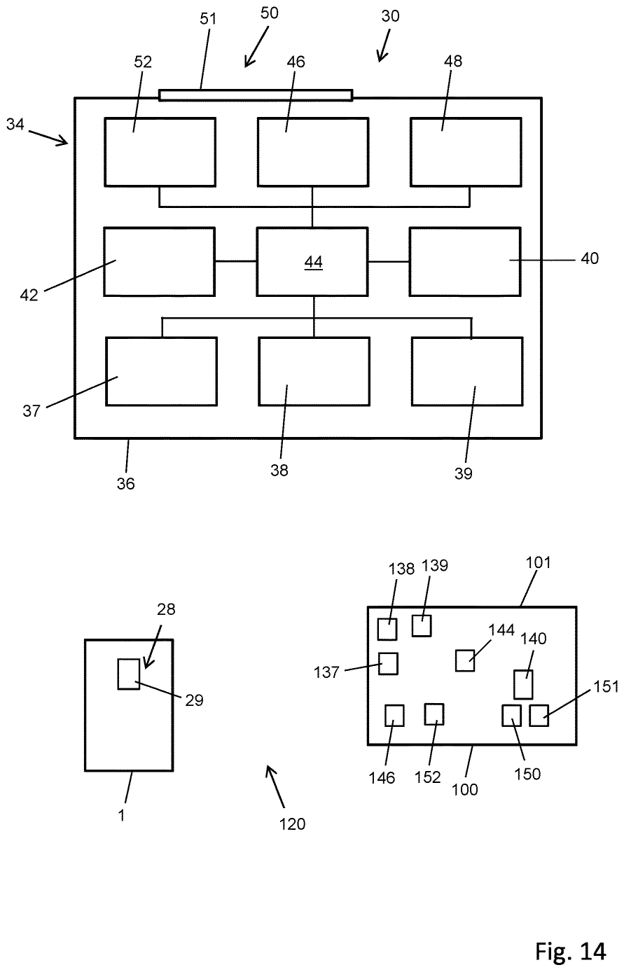

The add-on device 30 as schematically illustrated in FIG. 14 comprises an electronic module 34. The electronic module 34 comprises a printed circuit board 36. The electronic module 34 comprises a processor 44, an electronic memory 40 and a clock 42. Furthermore, the electronic module 34 comprises a power source 46 and the sensor 48. The electronic module 34 also comprises a signal generator 52 coupled to the processor 44 and/or coupled to the power source 46. The electronic module 34 further comprises at least one of a reader 37 and a wireless transceiver 38, 39. The add-on device 30 also comprises a user perceptible device identification 50.

With some examples the device identification 50 comprises or includes the signal generator 52 coupled to the processor 44 and operable or reconfigurable by the processor 44. With some examples the device identification 50 comprises an electronic display 51 operable to visibly illustrate various identifiers 55 being indicative of the injection device 1 to which the add-on device 30 is connected or coupled to.

With some examples the signal generator 52 is operable to generate at least one of a visual, an acoustic and a haptic device identification and hence produce or to generate a visual identifier, an acoustic identifier or a haptic identifier.

With some examples the add-on device 30 comprises numerous signal generators 52. Here, a first signal generator may be operable to generate a visual device identification or a visual identifier. Another electronic signal generator 52 may be operable to generate an acoustic identifier and/or a further signal generator 52 may be operable and configured to generate a haptic identifier.

With the example of FIG. 2 the signal generator 52 may comprise a speaker operable to generate an acoustic signal, wherein the acoustic signal represents or constitutes an identifier, e.g. in form of an acoustic code or sequence being indicative of the injection device 1 or of the medicament 24 located inside a medicament container 21 of the injection device 1.

The electronic signal generator 52 is coupled to the processor 44 of the add-on device 30 and in particular of the electronic module 34 of the add-on device 30. The processor 44 is configured to generate different acoustic signals in cooperation with the signal generator 52 implemented as a speaker. Here, a first acoustic signal being indicative of a first injection device 1 or medicament 24 and at least a second acoustic signal being indicative of another injection device 1′ or another medicament 24′ can be generated. Hence, the first and second acoustic signals are audibly distinguishable by a user of the injection device 1.

With the example of FIG. 3 the movable part 70 forms or constitutes the reconfigurable and user-perceptible device identification 50. In FIG. 3 there are illustrated three different configurations of the reconfigurable device identification 50. When or during the add-on device 30 is attached to a first injection device 1 the add-on device 30 is configured to read a machine-readable identification 28 of the injection device 1. The machine-readable identification 28 may be implemented as an electronic identifier 29 as illustrated in FIG. 14 and may be configured to communicate with a suitable reader 37, e.g. with a short range transceiver 38 of the add-on device 30. The transceiver 38 being operable to read-out the machine-readable identification 28 of the injection device 1.

The reader 37 or transceiver 38 is operably connected to the processor 44 and is hence operable to transfer the data obtained from the machine-readable identification 28 to the processor 44. The processor 44 is in turn operable to process the machine-readable information as obtained from the machine-readable identification 28 and is further operable to configure or to reconfigure the user perceptible device identification 50. With the example as illustrated in FIG. 3 the user perceptible device identification 50 comprises a signal generator 52, e.g.

implemented as a light source operable to illuminate the movable part 70. Here, the movable part 70 may comprise at least one of a window or a transparent portion through which an optical signal as generated by a light source of the electronic signal generator 52 becomes visible.

Also, the entirety of the movable part protruding from the device body 60 may be of a transparent or semitransparent material. It may comprise a frosted or translucent plastic material, e.g. providing an optical diffusion when illuminated by a light source. This way, the movable part may be homogenously illuminated.

A configuration or reconfiguration of the user-perceptible device identification 50 is typically conducted in the course of assembly of the add-on device 30 to the injection device 1. With the example as illustrated on the left of FIG. 3 the add-on device 30 is attached to a first injection device 1 equipped with a first machine-readable identification 28. The reader 37 or the transceiver 38 of the add-on device 30 is operable to read-out the machine-readable identification 28 being indicative of the type of the injection device 1 or being indicative of the medicament 24 located inside the injection device 1. In response to a read-out of machine-readable identification 28 the processor 44 of the add-on device 30 obtains the characteristic information of the injection device 1 as provided by the machine-readable identification 28. Then and in response to receive the machine-readable identification data the processor 44 is operable to conduct a reconfiguration of the user-perceptible device identification 50, which may then produce or generate a first identifier 55 that is visible at or through the movable part 70. With an example as illustrated in FIG. 3 the movable part 70 may be illuminated in a first color thus forming or constituting the first identifier 55.

In the middle illustration of FIG. 3 the add-on device 30′ is attached to another injection device 1′ equipped with another machine-readable identification 28′. Accordingly, and in response to a read-out of the machine-readable identification 28′ the processor 44 reconfigures the user perceptible device identification 50, which then generates a second identifier 55′. The second identifier 55′ may distinguish from the first identifier 55 by its color.

With a further example as illustrated to the right in FIG. 3 the add-on device 30″ is attached to another injection device 1″, distinguishes from the injection devices 1, 1′ or from the medicament 24 located inside the respective injection device 1, 1′. In addition or alternatively, the injection device 1″ may distinguish with regard to its operability from the injection devices 1, 1′.

The add-on device 30″ is configured to read-out the machine-readable identification 28″ and to reconfigure its user-perceptible device identification 5ß thereby generating a third identifier 55″, which distinguishes from the other identifiers 55, 55′. Again, the identifier 55″ may distinguish from the other identifiers 55, 55′ through its color.

With the further example of FIG. 4 a similar reconfiguration of the add-on device 30 is implemented. Here, the add-on device 30 comprises a user-perceptible device identification 50 implemented as a transparent or semitransparent window 56, which is integrated into the sidewall 61 of the device body 60. The window 56 may be provided at or near a proximal end of the sidewall 61. The window or window section 56 may comprise an annular shape. It may be made of a transparent or semitransparent, e.g. a frosted and/or translucent plastic material, e.g., comprising or forming a light guide or light guiding structure optically coupled to the electronic signal generator 52, which comprises at least one or several light sources.

As illustrated in the left section of FIG. 4 when the add-on device 30 is attached to a first injection device 1 the user perceptible device identification 50 and hence the window section 56 is operable to generate a first identifier 55, characterized by a first color.

In another configuration of the add-on device 30′ when the add-on device 30 is attached to a different type of an injection device 1′ equipped with a different machine-readable identification 28′ the user perceptible device identification 50 is reconfigured. Accordingly, the window 56 forms or constitutes a second identifier 55′. For instance, the window 56 appears in a second color.

When attaching the add-on device 30″ to a further add-on device 1″ and upon read-out of a respective machine-readable a modification 28″ the device identification 50 produces or generates a third identifier 55″. Here, the window 56 appears in a third color that distinguishes from the colors of the identifiers 55, 55′.

As further illustrated in FIG. 5 with the example of an injection system 120 the add-on device 30 attached to the injection device 1 may be in a first configuration thereby generating a first identifier 55 on or through the movable part 70. Here, the movable part 70 may be illuminated in a color that is substantially identical to the color of a label 17 of the body 6 of the injection device 1. The label 17 as provided on the outside surface of the sidewall of the body 6 may distinguish from other devices simply by the color of the body 6. With other examples the label 17 may be a printed label. The label 17 may represent an identification 28 and hence a machine-readable identification 28 of the injection device, e.g. in form of an optical or visual code.

With some examples and when the label 17 and hence the machine-readable identification 28 is a color of a portion of the housing of the injection device 1 it may be beneficial when the user perceptible device identification 50 of the add-on device 30 is illuminated or reproduced in the same color as the color of the label 17.

With some examples the machine-readable identification 28, e.g. implemented as an electronic identifier readable by the reader 37 or by the short range transceiver 38 of the add-on device 30 may simply contain a code representing a color of the label 17. As further illustrated in FIGS. 5 and 14 the injection system 120 comprises the injection device 1 and the add-on device 30.

Optionally, the injection system 120 may further comprise an external electronic device 100. The external electronic device 100 comprises a housing 101 as illustrated in FIG. 8. The electronic device 100 may be implemented as a portable electronic device. It may comprise a smartphone, a smart watch or a tablet computer. The external electronic device 100 typically comprises a device processor 144 coupled to an electronic device memory 140. The electronic device 100 further comprises a device power source 146. Also, the electronic device 100 comprises a device display 151, typically implemented as a touch sensitive display. The device display 151 may be operable to provide or to emulate a device signal generator 152. Hence, the device display 151 is operable to provide at least one of an illustration 153 and a notification 154 to a user.

With some examples the external electronic device 100 is operable to generate a device identification 150 being indicative of the injection device 1 currently in use or currently connected to the add-on device 30. As particularly illustrated in FIG. 14 the external electronic device 100 comprises at least one of a device reader 137 and 8 device transceiver 138, 139. The device transceiver 138 may be implemented as a short range wireless transceiver. The transceiver 139 may be implemented as a local range wireless transceiver.

The device transceiver 138 is operable to communicate with the transceiver 38 of the add-on device. The transceiver 139 is configured to communicate with the transceiver 39 of the add-on device 30. The reader 137 may be operable to read a visual identifier as provided on the injection device 1. With some examples the device reader 137 comprises an imaging system. It may comprise a camera objective and a spatially resolving detector in order to read or to capture a visual code, e.g. provided on an outside surface of the injection device 1 or as provided on an outside surface of the medicament container 21.

The external device 100 is configured to set up a communication link, e.g. a wireless communication link with the add-on device 30. For this, the local range transceiver 39 of the add-on device 30 may communicate wirelessly with the local range device transceiver 139 of the add-on device 100. Once a communication link has been established between the electronic device 100 and the add-on device 30 the electronic device 100 may be operable to visibly illustrate a device identification 150 on the device display 151. The device identification 150 as shown on the device display 151 may correspond to the device identification 50 or the identifier 55 as reproduced or provided by the add-on device 30. With some examples the color of the identifier 55 as generated by the device identification 50 may correspond to or may be equal to the color of the device identification 150 as reproduced on the display 151 of the external electronic device 100.

The electronic device 100 is typically implemented as a handheld electronic device. It can be held in a hand 110 of a user. With further examples (not illustrated) the external electronic device 100 may be worn on the wrist 111 of a user. With the example as illustrated in FIG. 5 the electronic device 100 is held in a palm 112 of the user. The device display 151 is implemented as a touch sensitive display and can be operated by a thumb 114 and/or by various fingers 116 of the user's hand 110.

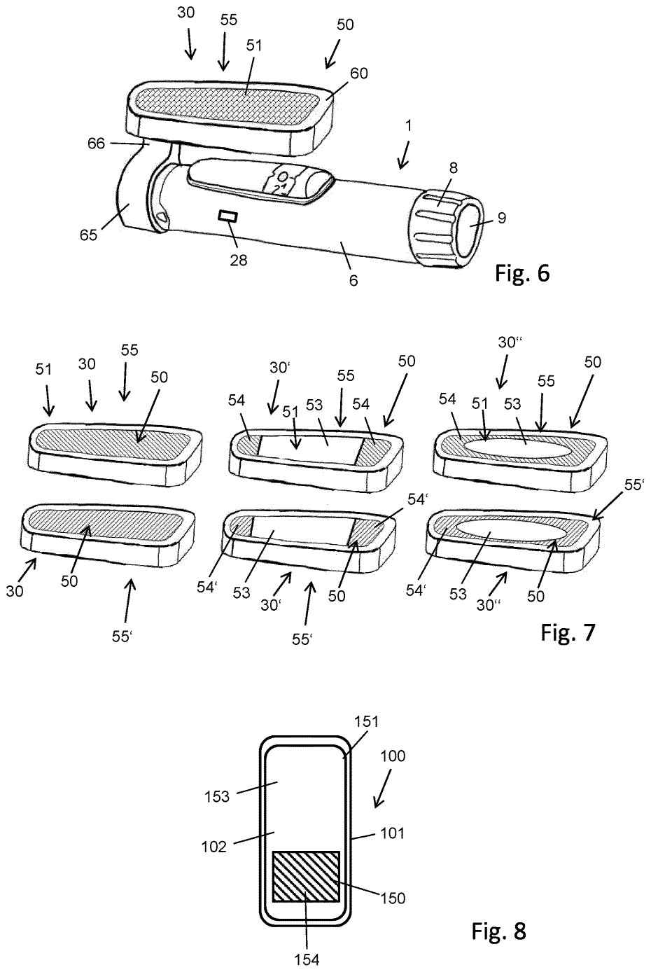

In FIGS. 6 and 7 there is illustrated another example of an add-on device 30. The add-on device 30 also comprises a device body 60, which now comprises an own display 51. The add-on device 30 as illustrated in FIGS. 6 and 7 comprises a fastener 65, e.g. in form of a mount configured to attach to a distal end of the body 6 of the injection device 1. The add-on device 30 may be mounted in the interface between the cartridge holder 7 and the body 6 of the injection device 1. The fastener 65, e.g. in form of an annular or clip-like mount is rigidly connected to the planar-shaped display 51 through a neck portion 66 protruding radially outwardly from the sidewall of the body 6 when the add-on device 30 is suitably attached to the injection device 1. The display 51 is of rather planar shape and extends parallel to the longitudinal direction of the body 6.

With the example of FIG. 6 the user-perceptible device identification 50 is provided by the electronic display 51 of the add-on device 30. Here, a color of the display 51, e.g. a background color of the display 51 may be indicative of the machine-readable identification 28 of the injection device 1.

As illustrated in FIG. 7 and with a first example of the add-on device 30 the entirety of the display 51 may be configured to reproduce or to generate a first identifier 55 or a second identifier 55′. The first identifier 55 and the second identifier 55′ distinguish from each other by their color. With the example of the add-on device 30 reproduced on the left of FIG. 1 the identifiers 55, 55′ distinguish by their color. Here, the machine-readable identification can be reproduced on the display 51 by a variation of the background color of the display 51.

With the further example as reproduced in the middle section of FIG. 7 the add-on device 30′ comprises a display 51 separated in different section 53, 54. The first display section 53 is operable to visibly illustrate some injection device-related parameters or may provide readable notifications to the user. The display sections 54 represent the first identifier 55 or a second identifier 55′. With the first identifier 55 of the add-on device 30′ the display section 54 is reproduced in a first color. With the second identifier 55′ of the add-on device 30′ the display section 54′ is reproduced in second, hence in a different color.

With a further example of the reconfigurable display 51 as illustrated on the right in FIG. 7 the display 51 is separated into first display section 53 surrounded by a second display section 54. The first display section 53 is configured to visually illustrate various device parameters of the injection device 1 and/or of the add-on device 30. The surrounding second display section 54 is operable to generate or to reproduce the variable identifier 55, 55′. With the add-on device 30″ and in a first configuration the second display section 54 is configured to generate a first identifier 55. Here, the second display section 54 may be provided in a first color. With another configuration of the add-on device 30″ the identifier 55′ changes. Here, the display section 54′ is e.g. illuminated in a color that distinguishes from the color of the display section 54 of the add-on device 30″.

The examples of FIGS. 3-7 show the reconfigurable user perceptible device identification 50 that is operable to change its visual appearance. In the same or like manner the device identification 50 may be equipped with an electronic signal generator 52, e.g. implemented as a speaker or as a vibration module by way of which a characteristic acoustic sequence or vibration sequence could be generated that is perceivable by the user in a similar or like manner compared to the varying visual appearances, that may distinguish by their color of that may distinguish by a visible pattern or the like visual illustration.

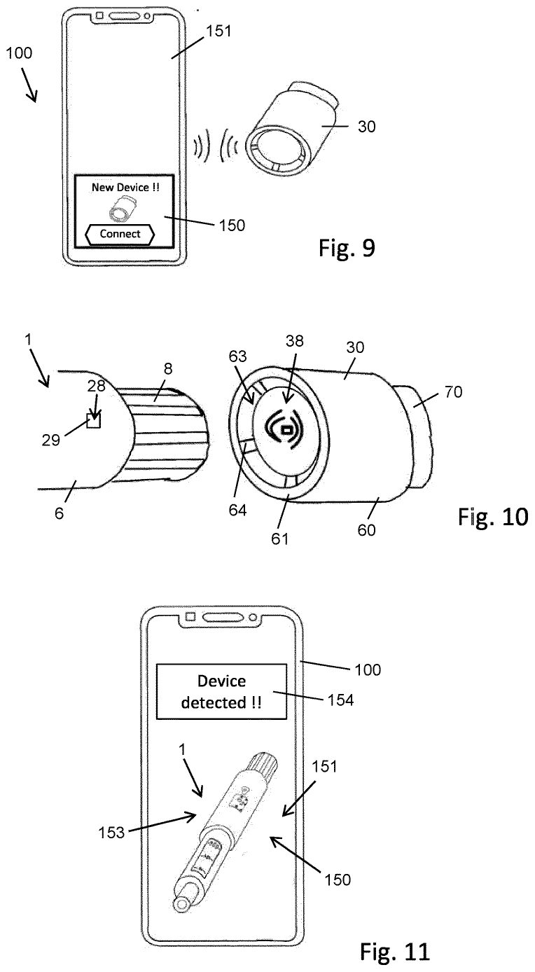

As further illustrated in FIG. 9 and upon use of the add-on device 30 with an injection device 1 the add-on device 30 may be wirelessly paired with the external electronic device 100. Here, a wireless pairing routine may be conducted or initiated by the external electronic device 100. As an example, the local range device transceiver 139 of the external electronic device 100 may establish a communication link with the local range transceiver 39 of the add-on device 30.

Here, the external electronic device 100 may be paired with the add-on device 30 by way of a Bluetooth connection or by a Bluetooth low energy communication link. Once a communication link has been established the external electronic device 100 may be operable to visibly illustrate a device identification 150 on the device display 151 as illustrated in FIG. 9. Here, detection of the presence of an add-on device 30 in the transmission range of the external electronic device 100 is indicated to a user. The user may prompt or may trigger a pairing of the add-on device 30 with the external electronic device 100 by pressing a respective connection button as emulated or illustrated on the device display 151.

Thereafter or even before a pairing of the add-on device 30 with the external electronic device 100 the add-on device 30 is to be connected with the injection device 1 as illustrated in FIG. 10. Before or during mounting of the add-on device 30 onto the dose dial 8 at the proximal end of the injection device 1 the short range transceiver 38 of the add-on device 30 reads the machine-readable identification 28 as provided on or in the injection device 1.

For this it may be beneficial when the machine-readable identification 28 comprises an electronic identifier 29, such as a NFC tag that is readable by the short range transceiver 38 of the add-on device 30. Upon readout of the machine-readable identification 28, which may be located inside or on an outside of the injection device 1 characteristic information being indicative of the type of the injection device 1 and/or of the type of the medicament 24 to be dispensed by the injection device 1 can be provided and transferred to the add-on device 30. In accordance to the data gathered from the machine-readable identification 28 the processor 44 of the add-on device 30 is operable to configure or to reconfigure the user perceptible device identification 50.

The pairing of the add-on device 30 with the injection device 1 as well as the readout of the machine-readable identification 28 conducted by the add-on device 30 may be also transmitted to the external electronic device 100, which in turn may indicate to a user that the add-on device 30 has been suitably paired and/or mechanically connected to the injection device 1.

As illustrated in FIG. 11 the electronic device display 151 may provide an illustration 153 of the injection device 1. It may further provide a notification 154 to the user, that the add-on device 30 detected a particular type of an injection device 1 and/or that the add-on device 30 has read-out the machine-readable identification 28 of this particular injection device 1. When the add-on device 30 has been correctly assembled to the injection device 1 and upon configuring or reconfiguring the user-perceptible device identification the add-on device 30 produces or generates a permanent, persistent or a temporal identifier 55 that is perceivable by the user and which characterizes the injection device 1 in accordance to the machine-readable identification 28.

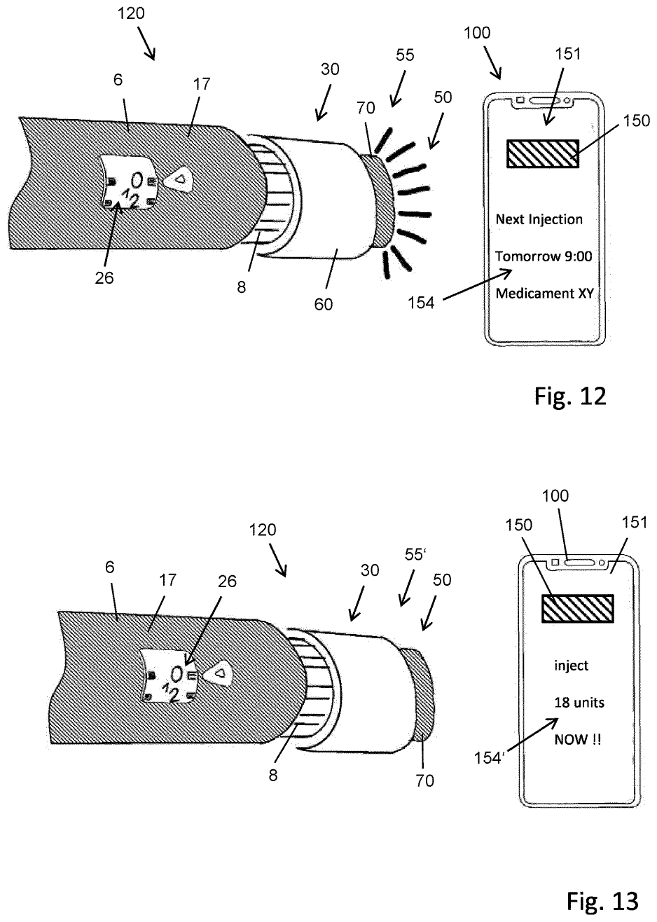

With the example as shown in FIG. 12 the device identification 55 is integrated into the movable part 70 of the add-on device 30 and is operable to provide a visible identifier 55 in form of illuminating the movable part 70 in a characteristic color or by producing a characteristic temporal illumination sequence.

With some examples the color of the movable part 70 and hence the identifier 55 may comprise the same or the like color as a label 17 provided on an outside surface of the body 6 of the injection device. The same color may be reproduced as a device identification 150 on the display 151 of the external electronic device 100. In addition, the external electronic device 100 may provide a notification 154 comprising instructions with regard to the operation of the injection device or with regards to a prescribed medication schedule.

With FIG. 13 a further configuration of the injection system 120 is illustrated. Here, the movable part 70 may still be illuminated in a characteristic color. Here, the notification 154 of the external electronic device 100 has changed to a notification 154′. In accordance to a prescribed medication schedule the external electronic device 100 informs the user to conduct an injection procedure right now. The external electronic device 100 also provides information about the dosing of the medication. In addition to the notification 154′ as provided on the electronic device display 151 the communication link between the external electronic device 100 and the add-on device 30 may be further used to indicate to a user that an injection procedure is due, e.g. by a simple modification of the identifier 55′.

Here, the identifier 55′ may comprise a flashlight or a blinking light. In addition, and when equipped with an additional electronic signal generator 52, e.g. implemented as acoustic or haptic signal generator the external electronic device 100 may invoke generation of an acoustic signal or of a haptic signal as an identifier 55 on or by the add-on device 30.

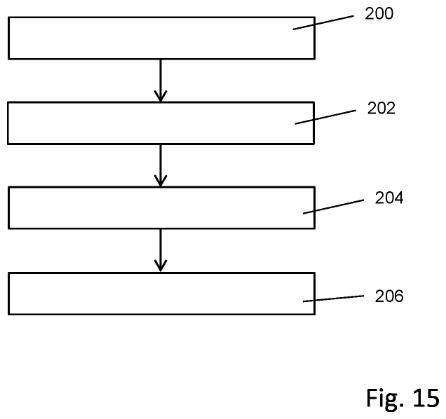

In FIG. 15 there is illustrated a flowchart of a method of configuring the add-on device 30. In a first step 200 an injection device 1 as described above is provided. The injection device 1 comprises a machine-readable identification 28 being indicative of the type of the injection device 1 and/or of a type of the medicament 24 located inside the injection device 1 or intended to be injected with the injection device 1.

In a subsequent step 202 there is also provided an add-on device 30 as illustrated and described above. In step 204 the machine-readable identification 28 is readout either by the add-on device 30 or by an optional external electronic device 100. Based on the information obtained from the machine-readable identification 28 the user-perceptible device identification 50 of the add-on device 30 is configured or reconfigured so as to durably or temporally adapt to the specific type of the injection device 1.

With some examples the device identification 50 is configured to permanently remain in the configured or reconfigured state. Accordingly, the device identification 50 may permanently remain in the configured state and may permanently reflect or represent the injection device 1 to which the add-on device 30 is attached to. Hence, in step 206 the user perceptible device identification 50 of the add-on device 30 is reconfigured or configured in accordance to the machine-readable identification 28. With some examples the device identification 50 is operable to provide at least a first and a second identifier 55, 55′, wherein the first identifier 55 distinguishes from the second identifier 55′ by its color.

With some examples readout of the machine-readable identification 28 is conducted by a reader 37 and/or by a transceiver 38, 39 of the add-on device 30. With other examples the readout of the machine-readable identification to 28 is conducted by the external electronic device 100.

The respective information of the machine-readable identification 28 is then transferred to the add-on device 30 via a communication link between the add-on device 30 and the external electronic device 100.

With some examples the display 51 comprises an electrophoretic display. The electrophoretic display may be reconfigurable to appear in different colors with a minimum of energy consumption.

### REFERENCE NUMBERS

1 injection device
2 distal direction
3 proximal direction
4 dose incrementing direction
5 dose decrementing direction
6 body
7 cartridge holder
8 dose dial
9 trigger
10 housing
11 connector
12 needle assembly
13 injection needle
14 inner needle cap
15 outer needle cap
16 protective cap
17 label
18 piston
19 piston rod
20 drive mechanism
21 medicament container
22 barrel
23 seal
24 medicament
25 outlet
26 window
27 dial extension
28 identification
29 electronic identifier
30 add-on device
34 electronic module
36 printed circuit board
37 reader
38 transceiver
39 transceiver
40 memory
42 clock
44 processor
46 power source
48 sensor
50 identification
51 display
52 signal generator
53 display section
54 display section
55 identifier
56 window
60 device body
61 sidewall
62 flange portion
63 receptacle
64 fastening rib
65 fastener
66 neck portion
70 movable part
100 electronic device
101 housing
102 display
110 hand
111 wrist
112 palm
114 thumb
116 finger
120 injection system
137 device reader
138 device transceiver
139 device transceiver
140 device memory
144 device processor
146 device power source
150 device identification
151 device display
152 device signal generator
153 illustration
154 notification

1-17. (canceled)

18. An add-on device for attaching to an injection device comprising a machine-readable identification, the add-on device comprises:
a device body fastenable to a portion of the injection device,
a reconfigurable and user-perceptible device identification, and
an electronic module comprising a processor coupled to the device identification and operable to reconfigure the device identification in accordance with the machine-readable identification of the injection device.

19. The add-on device according to claim 18, further comprising at least one of a reader and a transceiver coupled to the processor and operable to read or to obtain the machine-readable identification of the injection device.

20. The add-on device according to claim 19, wherein the at least one of the reader and the transceiver comprises a wireless near field transceiver operable to wirelessly communicate with the machine-readable identification of the injection device to read or to obtain information or data provided by or in the machine-readable identification of the injection device.

21. The add-on device according to claim 19, wherein the at least one of the reader and the transceiver comprises a wireless local range transceiver operable to wirelessly communicate with an external electronic device to receive the machine-readable identification of the injection device from or via the external electronic device.

22. The add-on device according to claim 18, wherein the device identification in a first configuration represents a first identifier being indicative of a first machine-readable identification and wherein the device identification in a second configuration represents a second identifier being indicative of a second machine-readable identification, wherein the first identifier and the second identifier perceptibly distinguish from each other.

23. The add-on device according to claim 18, wherein the device identification comprises an electronic signal generator controlled or operable by the processor.

24. The add-on device according to claim 23, wherein the electronic signal generator is operable to generate at least one of a visual, an acoustic and a haptic device identification.

25. The add-on device according to claim 23, wherein the electronic signal generator is operable to generate various device identifications or various identifiers that distinguish by at least one of a color, a brightness and a temporal variation.

26. The add-on device according to claim 23, wherein the electronic signal generator comprises at least two light sources of different color to generate device identifications that distinguish with regard to their color.

27. The add-on device according to claim 18, further comprising an electronic display coupled to the processor and operable to illustrate the device identification.

28. The add-on device according to claim 18, wherein the device identification is persistently and/or durably perceivable from outside the add-on device.

29. The add-on device according to claim 18, wherein the device identification is arranged on or integrated into the device body.

30. The add-on device according to claim 18, further comprising a movable part operably couplable with a trigger of the injection device and movable relative to the device body (60), wherein the device identification is arranged on or integrated into the movable part.

31. An injection system comprising:
an injection device for setting and injecting a dose of a medicament and comprising a machine-readable identification being indicative of at least one of a type of the injection device and the type of the medicament, and
an add-on device according to claim 18 detachably attachable to the injection device.

32. The injection system according to claim 31, further comprising an external electronic device operable to read the machine-readable identification of the injection device and operable to transmit the identification of the injection device to the add-on device.

33. A method of configuring an add-on device, the method comprising the steps of:
providing an injection device comprising a machine-readable identification,
providing an add-on device for attaching to an injection device comprising a machine-readable identification, the add-on device comprises:
a device body fastenable to a portion of the injection device,
a reconfigurable and user-perceptible device identification, and
an electronic module comprising a processor coupled to the device identification and operable to reconfigure the device identification in accordance with the machine-readable identification of the injection device.
reading of the machine-readable identification, and
reconfiguring the user-perceptible device identification of the add-on device in accordance with the machine-readable identification.

34. A computer program comprising computer readable instructions, which when executed by the processor of the add-on device of claim 18 cause the processor to reconfigure the user-perceptible device identification in accordance with the machine-readable identification of the injection device.
